(19) United States
(12) Patent Application Publication
(10) Pub. No.: US 2026/0014023 A1
(43) Pub. Date: Jan. 15, 2026

(54) OPHTHALMIC SYSTEMS AND METHOD FOR ILLUMINATING A VITREOUS CAVITY

(71) Applicant: Alcon Inc., Fribourg (CH)

(72) Inventor: Paul R. Hallen, Colleyville, TX (US)

(21) Appl. No.: 19/256,449

(22) Filed: Jul. 1, 2025

Related U.S. Application Data
(60) Provisional application No. 63/670,227, filed on Jul. 12, 2024.

(51) Int. Cl. A61F 9/008

(52) U.S. Cl. CPC A61F 9/008; A61F 2009/00874

(57) ABSTRACT

In certain embodiments, an ophthalmic system is provided. The ophthalmic system includes a low power laser source configured to generate a low power laser beam, wherein the low power laser source has a power of between 5 milliwatts (mW) and 20 mW, and a first illumination device configured to transmit the low power laser beam into a vitreous cavity of an eye. The ophthalmic system further includes a red-green-blue (RGB) light-emitting diode (LED) light source configured to generate a RGB light, and a second illumination device configured to transmit the RGB light into the vitreous cavity of the eye.

### INTRODUCTION

Microsurgical procedures frequently involve precision cutting and/or removing of various body tissues. For example, certain ophthalmic surgical procedures may involve cutting and removing portions of the vitreous humor, a transparent jelly-like material that fills the posterior segment of the eye. The vitreous humor, or vitreous, is composed of numerous microscopic fibrils that are often attached to the retina. Therefore, cutting and removing the vitreous are performed with great care to avoid traction on the retina, the separation of the retina from the choroid, a retinal tear, or, in the worst case, cutting and removal of the retina itself.

When removing vitreous, ophthalmic surgeons typically utilize a vitrectomy handpiece to cut and suction the vitreous out of the eye, and an illumination device to illuminate the vitreous within the eye. However, because the vitreous is a substantially transparent gel-like material, it may lack contrast with other fluids and/or surrounding tissue in the eye. As such, the illumination device may struggle to adequately illuminate the vitreous, making it difficult for the surgeon to see and remove the vitreous within the eye. Such restrictions limit the efficiency of vitreous removal and may increase the amount of time needed to complete ophthalmic procedures, which increases patient risk.

### BRIEF SUMMARY

The present disclosure relates generally to an ophthalmic system and a method for illuminating a vitreous cavity of a patient during ophthalmic surgical procedures, such as vitreoretinal procedures.

In certain embodiments, an ophthalmic system is provided. The ophthalmic system includes a low power laser source configured to generate a low power laser beam, wherein the low power laser source has a power of between 5 milliwatts (mW) and 20 mW, and a first illumination device configured to transmit the low power laser beam into a vitreous cavity of an eye. The ophthalmic system further includes a red-green-blue (RGB) light-emitting diode (LED) light source configured to generate a RGB light, and a second illumination device configured to transmit the RGB light into the vitreous cavity of the eye.

In certain embodiments, a method for illuminating a vitreous cavity of a patient is provided. The method includes generating a low power laser beam for propagation in the vitreous cavity of the patient, generating a red-green-blue (RGB) light for propagation in the vitreous cavity of the patient, wherein the RGB light and the low power laser beam are generated simultaneously, and driving a vitrectomy subsystem for performing vitrectomy while the low power laser beam and the RGB light are simultaneously being generated. The low power laser beam is generated by a low power laser source that has a power of between 5 mW and 20 mW.

The following description and the related drawings set forth in detail certain illustrative features of one or more embodiments.

### BRIEF DESCRIPTION OF THE DRAWINGS

The appended figures depict certain aspects of the one or more embodiments and are therefore not to be considered limiting of the scope of this disclosure.

FIG. 1 illustrates an example operating environment for an ophthalmic surgical procedure, according to certain embodiments.

FIGS. 2A-2C illustrate cross-sectional side views of an eye and example illumination systems, according to certain embodiments.

FIG. 3A illustrates an example of the first illumination device shown in FIGS. 1 and 2A-2B with a diffusing surface, according to certain embodiments.

FIG. 3B illustrates an example of the first illumination device shown in FIGS. 1 and 2A-2B with a diffusing lens, according to certain embodiments.

FIG. 4 illustrates a block diagram of selected components of the illumination system of FIGS. 2A-2B, according to certain embodiments.

FIG. 5 illustrates a flowchart of a method for illuminating a vitreous cavity of a patient, according to an embodiment.

To facilitate understanding, identical reference numerals have been used, where possible, to designate identical elements that are common to the drawings. It is contemplated that elements and features of one embodiment may be beneficially incorporated in other embodiments without further recitation.

### DETAILED DESCRIPTION

It will be readily understood that the components of the embodiments as generally described herein and illustrated in the appended Figures can be arranged and designed in a wide variety of different configurations. Thus, the following more detailed description of various embodiments, as represented in the Figures, is not intended to limit the scope of the present disclosure but is merely representative of various embodiments. While the various aspects of the embodiments are presented in the Figures, the Figures are not necessarily drawn to scale unless specifically indicated.

Reference throughout this specification to the term “distal” refers to a system, device, component, end, portion, or segment that is disposed closer to a patient and/or further from a console during an ophthalmic procedure; and the term “proximal” refers to the system, device, component, end, portion, or segment that is disposed further from the patient and/or closer to the console during the ophthalmic procedure.

The vitreous body, often referred to as the vitreous humor or simply “the vitreous,” is a transparent, colorless, and gelatinous mass that fills the space between the lens and the retina of the eyeball (e.g., a posterior segment or inner portion of the eye). The vitreous makes up about 80% of the volume of the eyeball and helps maintain the round shape of the eye. Additionally, the vitreous assists in absorbing external mechanical shocks to the eye, provides nutrients to the lens, and supports the retina. The vitreous is mostly comprised of water with trace amounts of collagen and hyaluronic acid, which provide the vitreous with its gelatinous structure.

In vitreoretinal procedures (e.g., vitrectomy, retinal detachment repair, macular hole surgery, retinal laser surgery, epiretinal membrane (ERM) peel, etc.), the vitreous is often removed from the vitreous cavity of the eye to improve a surgeon's view of structures within the eye (e.g., the retina, macula, choroid, sclera, etc.). Removing the vitreous also helps provide the surgeon better access (e.g., less obstructed access) to the structures within the eye.

Current vitreoretinal surgeries often employ an ophthalmic surgical instrument, such as a vitrectomy probe, that is used to aspirate the vitreous out of the eye, and one or more illumination instruments for illuminating the vitreous cavity of the eye and the vitreous contained therein. For example, a surgeon may use one or more illumination devices such as, for example, an endoilluminator and/or a chandelier, to visualize the vitreous and the vitreous cavity of the eye during the surgery. However, illumination light provided by the endoilluminator and/or the chandelier may not effectively illuminate or allow the surgeon to visualize the vitreous within the eye because the vitreous is a substantially transparent gel-like material that lacks contrast with surrounding tissues within the eye. As such, because the surgeon may not be able to easily see the vitreous within the eye, it may be difficult to efficiently and/or completely remove the vitreous from within the eye.

To help remove the vitreous, fluid (e.g., a basic saline solution (BSS)) is often irrigated into, and aspirated out of the eye. However, for similar reasons as presented above, it is difficult for surgeons to visually differentiate between the fluid and the vitreous. Accordingly, a large volume of fluid (e.g., up to 100 cubic centimeters (cc) to 125 cc) is often exchanged to achieve adequate vitreous removal. Consequently, exchanging a large volume of fluid further limits the efficiency of the vitreoretinal surgery, and can result in unwanted and unintentional trauma to ocular tissues, which can lead to potentially permanent damage to the eye, disturbing its function and/or causing various other complications. As such, current vitreoretinal surgery techniques present a variety of limitations.

Accordingly, the embodiments described herein provide ophthalmic systems and illumination systems that provide improved visualization of vitreous within an eye. For example, each of the illumination systems herein combine a low power laser beam with a red-green-blue (RGB) light, which improves optical contrast between the vitreous and other fluids/surrounding tissues within the eye. The illumination systems described herein, therefore, improve the efficiency of vitreous removal, and reduce the duration of the surgery and the likelihood of unwanted and unintentional trauma to ocular tissues.

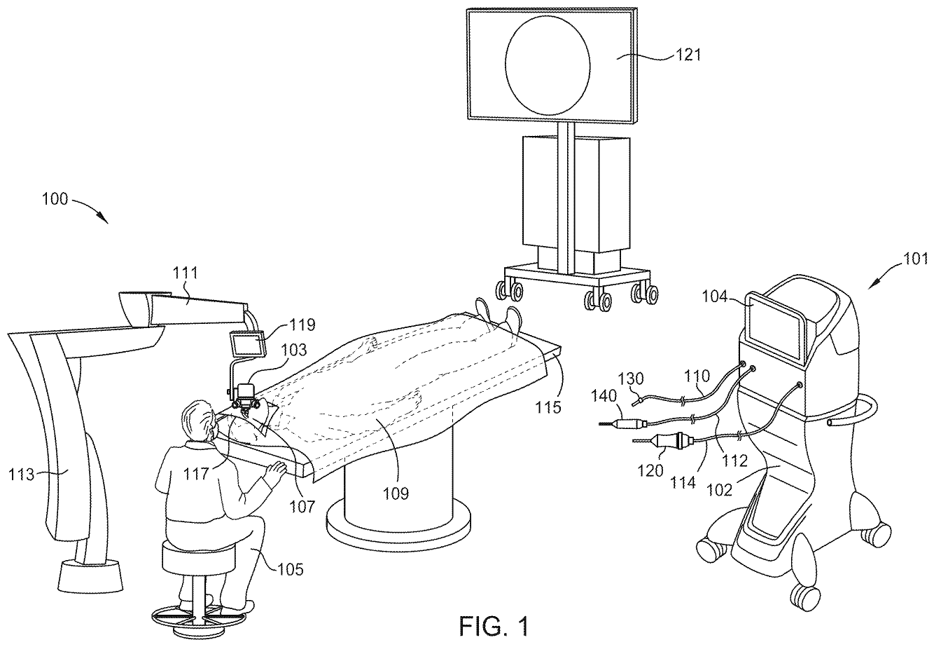

FIG. 1 illustrates an example operating environment 100 for an ophthalmic surgical procedure (e.g., a vitreoretinal procedure), according to certain embodiments. The operating environment 100 includes an ophthalmic surgical system 101 and an ophthalmic microscope 103 which may be used to perform ophthalmic procedures on an eye 107 of a medical patient 109. A surgeon 105 uses the ophthalmic surgical system 101 and the ophthalmic microscope 103 to visualize structures on and in the eye 107 during surgery. The ophthalmic microscope 103 is supported on, in this illustration, an adjustable overhead arm 111 of a microscope support pedestal 113. The patient 109 may be supported on an operating table 115. The ophthalmic microscope 103 is movable with the overhead arm 111 in three dimensions so that the surgeon 105 can position the ophthalmic microscope 103 as desired with respect to the eye 107 of the patient 109.

In certain embodiments, the ophthalmic microscope 103 comprises a high resolution, high contrast stereo viewing surgical microscope. The ophthalmic microscope 103 will often include a monocular eyepiece 117 or binocular eyepieces 117, through which the surgeon 105 will have an optically magnified view of the relevant eye structures that the surgeon 105 will need to see to accomplish a given surgery or diagnose an eye condition of the patient 109.

The ophthalmic microscope 103 includes a digital camera and broadband light source for capturing color (red, green, and blue) images, a multi-spectral imaging (MSI) device, and/or other type of imaging device. Digital images captured using the camera may be displayed on a display device within the ophthalmic microscope 103.

The ophthalmic microscope 103 may include two display devices that are viewable through binocular eyepieces 117 and that display images of the patient's eye 107 captured from different viewpoints by two cameras to provide stereoscopic viewing. For example, the ophthalmic microscope 103 may be implemented as the NGENUITY 3D VISUALIZATION SYSTEM provided by Alcon Inc. of Fort Worth Texas.

Images from the ophthalmic microscope 103 may be additionally or alternatively be displayed on one or more display devices. For example, the one or more display devices may include a display device 119 fastened to the supporting arm 111 above the ophthalmic microscope 103.

In order to relieve the surgeon 105 from the need to constantly look into the eyepieces 117 to obtain a stereoscopic view, the one or more display devices may include a display device 121 that may be implemented as a three-dimensional display device. The display device 121 may therefore provide a stereoscopic view of images captured using the ophthalmic microscope 103. The display device 121 may be embodied as any type of three-dimensional display device known in the art, including those that do or do not use special filtering glasses. For some types of three-dimensional display devices, the perception of three dimensions requires that the distance of the viewer from the display device 121 be within a threshold distance from the display device. The display device 121 may be mounted to a cart, a manually adjustable or robotic arm, or other manually or automatically adjustable support.

The ophthalmic surgical system 101 may be used by the surgeon 105 to perform the ophthalmic surgical procedure (e.g., the vitreoretinal procedure). The ophthalmic surgical system 101 includes a console 102 and an associated display 104. The display 104 may display, for example, data relating to system operation and/or system performance during a surgical procedure, which may be arranged in a graphical user interface (GUI). Generally, the console 102 includes one or more systems or subsystems that enable the surgeon 105 to perform ophthalmic surgical procedures. For example, the ophthalmic surgical system 101 also includes at least three ophthalmic surgical instruments (or handpieces) that are operably coupled to the systems or sub-systems of the console 102. The three ophthalmic surgical instruments include a vitrectomy probe 120, a first illumination device 130, and a second illumination device 140. In some embodiments, the ophthalmic surgical system 101 includes more than three instruments, and/or different types of instruments (e.g., a phacoemulsification probe, an irrigation probe, an aspiration probe, etc.).

The vitrectomy probe 120 may be used by the surgeon 105 to remove vitreous from the eye 107. The vitrectomy probe 120 is connected to the console 102 by a cable 114, which may include an aspiration line disposed therein, and may be coupled to one or more systems or subsystems included in the console 102. For example, the vitrectomy probe 120 may be coupled to a vitrectomy subsystem (e.g., vitrectomy subsystem 430) inside the console 102 that controls a pump and/or a vacuum for use in the removal of the vitreous. The vitrectomy subsystem may also provide power to the vitrectomy probe 120 and control the operations of the vitrectomy probe 120. In some implementations, the vitrectomy probe 120 may be a vitreous cutter, such as, for example, an oscillating vitreous cutter.

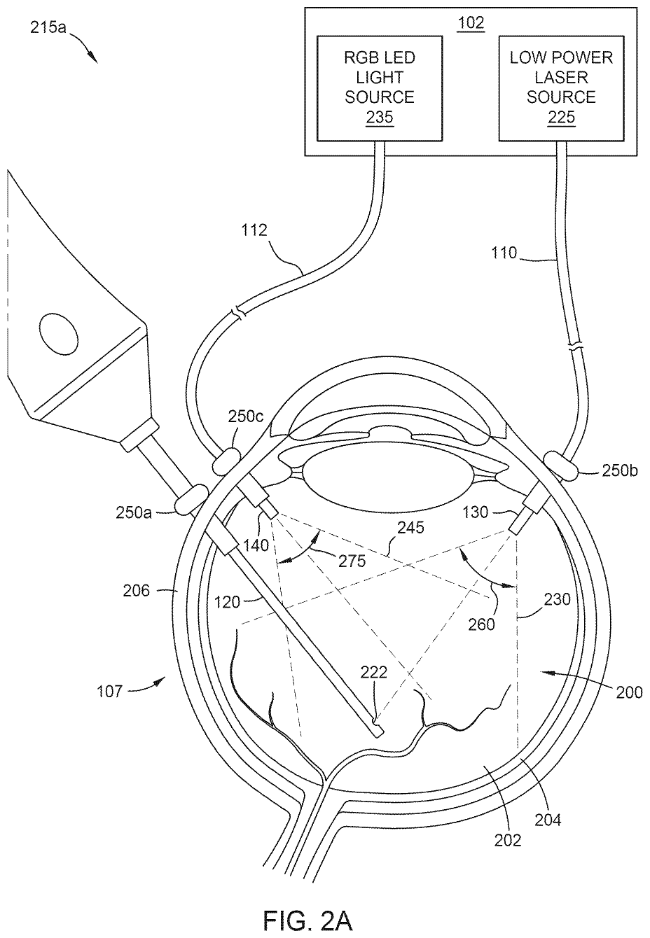

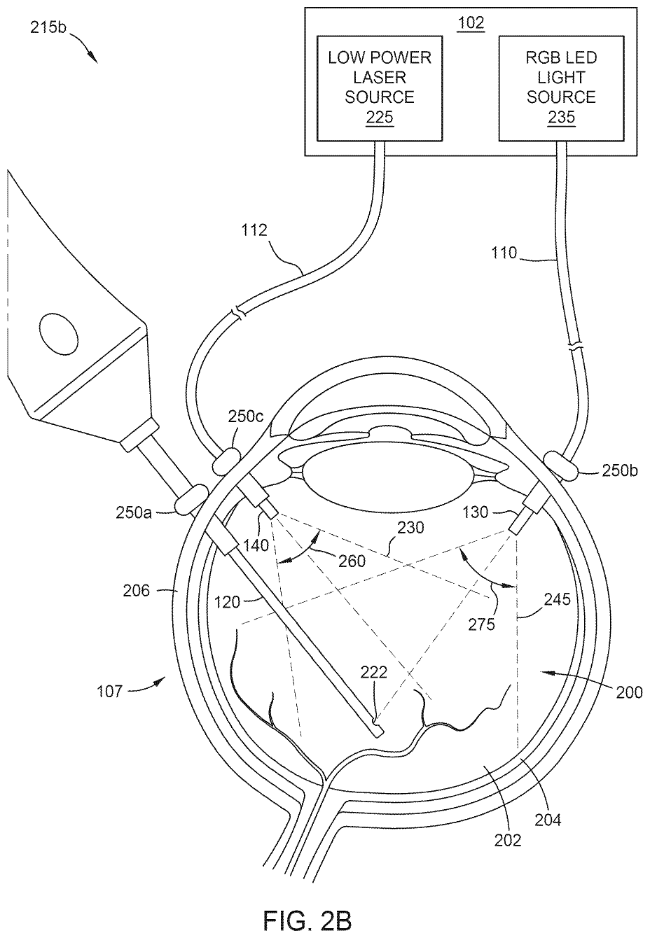

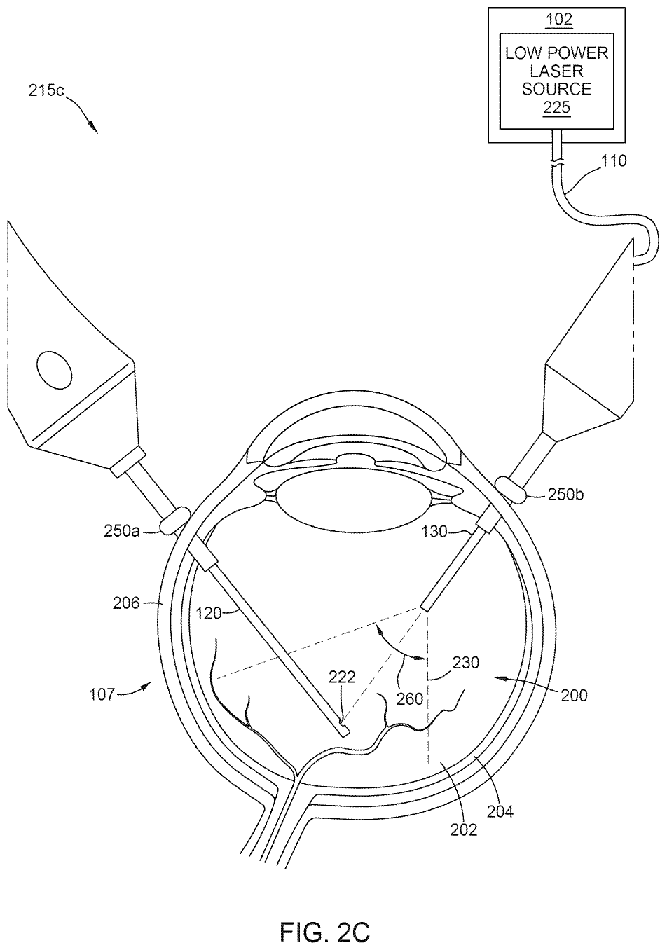

The first illumination device 130 and the second illumination device 140 are part of an illumination system (labeled 215 (215a and 215b) in FIGS. 2A-2B) that provides illumination light for illuminating the ocular space. As shown in FIG. 1, the first illumination device 130 is a chandelier and the second illumination device 140 is an endoilluminator; however, the first illumination device 130 may also be an endoilluminator and/or the second illumination device 140 may be a chandelier. The first illumination device 130 is connected to the console 102 via a first optical fiber cable 110, and the second illumination device 140 is connected to the console 102 via a second optical fiber cable 112. The first optical fiber cable 110 and the second optical fiber cable 112 each include an optical fiber disposed therein. The optical fiber is configured to transmit an LED light or a laser light generated by a light source or a laser source, respectively, that may be housed within the console 102. In some implementations, the LED light and/or the laser light may pass through one or more optical elements, such as, for example, one or more lenses, mirrors, and/or attenuators, before entering the optical fibers disposed in the first optical fiber cable 110 and the second optical fiber cable 112.

FIG. 2A illustrates a cross-sectional side view of the eye 107 and an example illumination system 215a, according to certain embodiments. The eye 107 includes a vitreous cavity 200 with vitreous 202, a retina 204, and a sclera 206. As shown in FIG. 2A, the vitrectomy probe 120, the first illumination device 130 (e.g., chandelier, endoilluminator, etc.), and the second illumination device 140 (e.g., chandelier, endoilluminator, etc.) are inserted into the eye 107. In particular, the vitrectomy probe 120 is inserted through a first cannula 250a, the first illumination device 130 is inserted through a second cannula 250b, and the second illumination device 140 is inserted through a third cannula 250c. The cannulas 250a-c may be used to create an incision through the sclera 206 of the eye 107. In some instances, the cannulas 250a-c may be inserted into an incision made by the surgeon 105 (or other medical professional) using another surgical tool.

Note that one or more of the illumination devices are illustrated herein in a genericized manner (i.e., without additional components). For example, a handpiece is typically used with an endoilluminator (the optical fiber running through the handpiece); however, the illumination devices here are shown generically for brevity.

The first illumination device 130 is connected to a low power laser source 225 via the first optical fiber cable 110, and the second illumination device 140 is connected to an RGB light-emitting diode (LED) light source 235 via the second optical fiber cable 112. The first illumination device 130, the second illumination device 140, the low power laser source 225, and the RGB LED light source 235 are each components of the example illumination system 215a.

The low power laser source 225 is disposed in the surgical console 102 and is configured to generate a low power laser beam, which is transmitted through the first optical fiber cable 110 to the first illumination device 130. As an example, the low power laser source 225 has a power of between 5 milliwatts (mW) and 20 mW (e.g., between 5.5 mW and 19.5 mW, 6 mW and 19 mW, 6.5 mW and 18.5 mW, or 7 mW and 18 mW).

The first illumination device 130 is configured to transmit the low power laser beam 230 generated by the low power laser source 225 into the vitreous cavity 200 of the eye 107. As an example, the first illumination device 130 is configured to transmit the low power laser beam 230 at a beam angle 260 that is less than or equal to 300° (degrees) (e.g., less than or equal to 280°, 260°, 240°, 220°, or) 200°.

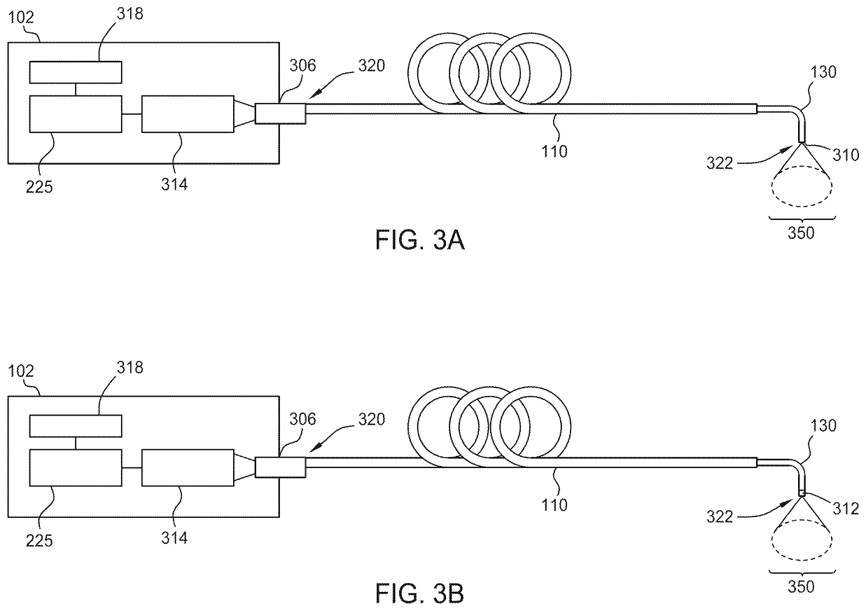

As described herein, the “low power laser beam” (e.g., low power laser beam 230) refers to a “speckled laser beam” that displays a granular or mottled pattern when projected onto a surface or material (e.g., vitreous 202) observed by the surgeon 105. The “speckled” nature of the low power laser beam 230 may occur due to interference of coherent light waves that have been scattered by a diffusing surface or lens. The diffusion of the low power laser beam 230 is described in more detail with reference to FIGS. 3A-3B.

Because the low power laser beam 230 is generated by the low power laser source 225, the low power laser beam 230 can be continuously transmitted into the vitreous cavity 200 for the duration of the ophthalmic procedure. In other words, the low power laser beam 230 does not harm or burn structures within the eye 107, and rather, is a non-focused laser light configured for illumination within the vitreous cavity 200. Accordingly, the low power laser beam 230 can be kept on throughout the ophthalmic procedure because it is transmitted at a power that is substantially less than a power of a laser used for treatment in laser-involved ophthalmic procedures.

The RGB LED light source 235 is disposed in the surgical console 102 and is configured to generate an RGB light 245. The RGB light 245 is a combination of one or more of a red light, a green light, or a blue light. Two or more of the red, green, and blue lights can be combined to produce a wide spectrum of colors, e.g., yellow, magenta, cyan, white, etc. The RGB light 245 generated by the RGB LED light source 235 is transmitted through the second optical fiber cable 112 to the second illumination device 140.

The second illumination device 140 is configured to transmit the RGB light 245 into the vitreous cavity 200. As an example, the second illumination device 140 is configured to transmit the RGB light 245 at a light angle 275 that is less than or equal to 300° (degrees) (e.g., less than or equal to 280°, 260°, 240°, 220°, or) 200°. Like the low power laser beam 230, the RGB light 245 is configured for illumination within the vitreous cavity 200, and can be continuously transmitted into the vitreous cavity 200 for the duration of the ophthalmic procedure.

During the ophthalmic procedure, the surgeon 105 may position the first illumination device 130 such that it is primarily directing the low power laser beam 230 at a port 222 of the vitrectomy probe 120. In some embodiments, the first illumination device 130 may be movable within the vitreous cavity 200, so that the surgeon 105 can change a direction of the low power laser beam 230 (e.g., to follow the port 222 of the vitrectomy probe).

In some embodiments, the first illumination device 130 may be fixed (or anchored) to the eye 107. As an example, when the first illumination device 130 is a chandelier, the chandelier can be fixed to the eye 107 via a self-retaining tip that anchors itself in the vitreous cavity 200 without additional support. As another example, the first illumination device 130 may be coupled to the second cannula 250b to fix the first illumination device 130 to the eye 107. That is, when the first illumination device 130 is a chandelier, the chandelier can be coupled to the second cannula 250b via a tight fit in the cannula 250b or via a locking mechanism. With the tight fit, the cannula itself provides a snug fit around the chandelier, holding it in place due to a pressure difference between the inside of the eye 107 and the external environment. With the locking mechanism, a clip or a latch secures the chandelier to the cannula, preventing it from moving during surgery. Fixing the first illumination device 130 to the eye 107 eliminates the need for the surgeon 105 to hold the first illumination device 130, affording greater freedom to operate on the patient 109.

The surgeon 105 may position the second illumination device 140 such that it is primarily directing the RGB light 245 to the front of the port 222 of the vitrectomy probe 120. Similar to the first illumination device 130, the second illumination device 140 may be movable within the vitreous cavity 200, or fixed to the eye 107 (e.g., via the third cannula 250c).

When the first illumination device 130 and the second illumination device 140 simultaneously transmit the low power laser beam 230 and the RGB light 245, respectively, within the vitreous cavity 200, a colored, speckled laser beam is produced where the low power laser beam 230 and the RGB light 245 combine or overlap. The colored speckled laser beam is absorbed (or picked up) by collagen fibers in the vitreous 202, which thereby illuminates the vitreous 202, making it more visible to the surgeon 105. Visualization of the vitreous 202 may also be improved due to an increase in optical contrast between the vitreous 202 and other fluid (e.g., BSS) and/or surrounding tissues (e.g., the retina 204) within the vitreous cavity 200.

As a result of the improved visualization of the vitreous 202, the surgeon 105 is able to more efficiently remove the vitreous 202 from the vitreous cavity 200. Additionally, the ratio of the vitreous 202 removed relative to other fluids (e.g., BSS) removed from the eye 107 increases due to the surgeon 105's ability to visibly identify when the vitreous 202 is entering the port 222 of the vitrectomy probe 120 (e.g., as opposed to when the BSS is entering the port 222). Thus, the amount of fluids exchanged in the eye 107 may be reduced, which reduces the risk of unwanted or unintentional trauma to ocular tissues, and allows the surgeon 105 to perform the ophthalmic surgical procedure in a shorter period of time, further reducing patient risk.

In some embodiments, a color and/or brightness of the RGB light 245 transmitted by the second illumination device 140 can be adjusted by the surgeon 105 (e.g., using the console 102) to maximize contrast and further improve visualization of the vitreous 202. For example, because the vitreous cavity 200 of the eye 107 is primarily a red-orange color, emitting a blue-green color with the RGB light 245 may be preferred because blue-green is opposite red-orange (according to a standard color wheel), and thereby provides the greatest amount of contrast. However, the color of the RGB light 245 may be adjusted according to each patient's needs, and/or the surgeon 105's preferences.

In some embodiments, the color and/or brightness of the RGB light 245 transmitted by the second illumination device 140 is kept the same throughout the ophthalmic procedure, but the color and/or brightness displayed to the surgeon 105 via the ophthalmic microscope 103, e.g., on the display devices 119, 121, can be adjusted by the surgeon 105. In other words, the color and/or brightness of the RGB light 245 displayed to the surgeon 105 can be different from an actual color and/or brightness of the RGB light 245 in the vitreous cavity 200. In some examples, such features can be achieved using visualization system, such as the NGENUITY 3D VISUALIZATION SYSTEM provided by Alcon Inc. of Fort Worth Texas.

FIG. 2B illustrates a cross-sectional side view of the eye 107 with another example illumination system 215b, according to certain embodiments. In FIG. 2B, the first illumination device 130 (e.g., chandelier, endoilluminator, etc.) is connected to the RGB LED light source 235 via the first optical fiber cable 110, and the second illumination device 140 (e.g., chandelier, endoilluminator, etc.) is connected to the low power laser source 225 via the second optical fiber cable 112. Accordingly, the first illumination device 130 transmits the RGB light 245, and the second illumination device 140 transmits the low power laser beam 230.

Note that, although shown in FIGS. 2A-2B as being disposed in the surgical console 102, the low power laser source 225 and/or the RGB LED light source 235 may also be disposed in the illumination devices 130, 140.

FIG. 2C illustrates a cross-sectional side view of the eye 107 with another example illumination system 215c, according to certain embodiments. In FIG. 2C, the first illumination device 130 is an illumination probe connected to the low power laser source 225 via an optical fiber cable 110, and transmits the low power laser beam 230 into the vitreous cavity 200.

In some embodiments, the first illumination device 130 transmits the low power laser beam 230 via a multi-core optical fiber disposed within the optical fiber cable 110. The multi-core optical fiber can include a plurality of inner core fibers (e.g., two inner core fibers, four inner core fibers, six inner core fibers, etc.) disposed inside an outer core fiber. Each of the inner core fibers may be contained within inner-core cladding, and the outer core fiber may be contained within an outer-core cladding. A proximal end of the multi-core optical fiber is connected to the console 102, and a distal end of the multi-core optical fiber is disposed at a distal end of the first illumination device 130. Accordingly, the low power laser source 225 generates the low power laser beam 230 and emits it onto the proximal end of the multi-core optical fiber. The low power laser beam 230 then propagates down a length of the inner cores and outer core, and is emitted from the distal end of the multi-core optical fiber.

In certain embodiments, because the low power laser beam 230 inherently provides sufficient illumination, the low power laser beam 230 may be utilized without RGB light. In other words, the surgeon 105 may be able to visualize the vitreous 202 using only the first illumination device 130. Thus, the illumination system 215c may not include the RGB LED light source 235.

However, in some other embodiments, the first illumination device 130 may be configured to propagate both the RGB light 245 as well as the low power laser beam 230, such that the low power laser beam 230 and the RGB light 245 are transmitted by a single illumination device. For example, the first illumination device 130 may include (1) a single fiber that can emit both beams, or (2) two separate fibers positioned next to each other, one for transmitting the RGB light 245 and the other for transmitting the low power laser beam 230. In other words, the first illumination device 130 and the second illumination device 140 can be the same device.

By using the illumination system 215c, the surgeon 105 can use one illumination device (e.g., the first illumination device 130) and one vitrectomy probe (e.g., vitrectomy probe 120) to visualize the vitreous 202 and perform a vitreoretinal procedure on the eye 107. As such, the surgeon 105 is able to hold the first illumination device 130 in one hand, and the vitrectomy probe 120 in the other hand, without needing to adjust or have an assistant hold another surgical instrument (e.g., a second illumination device) that is inserted in the eye 107. Accordingly, the illumination system 215c affords the surgeon 105 great freedom to operate while also reducing the number of incisions in the eye 107, which both lessen patient-risk.

FIG. 3A illustrates an example of the first illumination device 130 with a diffusing surface 310, according to certain embodiments. The first illumination device 130 is coupled to the console 102 via the first optical fiber cable 110, which includes one or more optical fibers disposed therein. A proximal end 320 of the first optical fiber cable 110 is coupled to the console 102 using a connector 306.

The console 102 provides a laser source, which in the example of FIG. 3A, includes the low power laser source 225 and an optical condensing element 314. The low power laser source 225 is in communication with a controller 318. In operation, upon receiving a control signal from the controller 318, the low power laser source 225 emits a laser beam that is then condensed and focused by the optical condensing element 314 on an opening exposes the proximal ends of one or more optical fibers extending through the first optical fiber cable 110.

The first illumination device 130 is then operable to transmit the laser beam received from the low power laser source 225 such that the low power laser beam 230 is projected through the diffusing surface 310 to illuminate an area 350, such as the vitreous cavity 200 during ophthalmic surgery. The diffusing surface 310 is disposed at a distal end 322 of the first illumination device 130, and is configured to diffuse the low power laser beam 230 transmitted by the first illumination device 130.

To diffuse the low power laser beam 230, the diffusing surface 310 may include one or more diffusing features. The diffusing features of the diffusing surface 310 may include a patterned surface, bumps or grooves which scatter the low power laser beam 230 in different directions, a coating (e.g., a diffusing film, frosted glass, or other similar diffusing material), etc. In some embodiments, the diffusing surface 310 and/or the distal end 322 may also be shaped into a certain structure (e.g., a bullet-like structure) which helps diffuse the low power laser beam 230. By diffusing the low power laser beam 230 using the diffusing surface 310, the low power laser beam 230 can be emitted in many directions, reducing its intensity and coherence in any single direction. Accordingly, the low power laser beam 230 is transmitted with a broader, more uniform distribution which improves the illumination of the vitreous 202 and the vitreous cavity 200.

The diffusing surface 310 may also be implemented at a distal end of the second illumination device 140. Further, in some embodiments, the diffusing surface 310 may be used to diffuse the RGB light 245 generated by the RGB LED light source 235.

FIG. 3B illustrates an example of the first illumination device 130 with a diffusing lens 312, according to certain embodiments. In FIG. 3B, diffusing lens 312 is disposed at the distal end 322 of the first illumination device 130 and is configured to diffuse the low power laser beam 230, e.g., similar to the diffusing surface 310 described with reference to FIG. 3A.

To diffuse the low power laser beam 230, the diffusing lens 312 may include one or more diffusing features. The diffusing features of the diffusing lens 312 may include a curved shape or other type of shape configured to scatter the low power laser beam 230 in a desired manner, an etched or frosted surface, a surface texturization (e.g., that includes microscopic irregularities), a gradient index (GRIN) design, a coating (e.g., a diffusing film, frosted glass, or other similar diffusing material), etc. Thus, the diffusing lens 312 helps the first illumination device 130 transmit the low power laser beam 230 with a broader, more uniform distribution which improves the illumination of the vitreous 202 and the vitreous cavity 200.

The diffusing lens 312 may also be implemented at a distal end of the second illumination device 240. Further, in some embodiments, the diffusing lens 312 may be used to diffuse the RGB light 245 generated by the RGB LED light source 235.

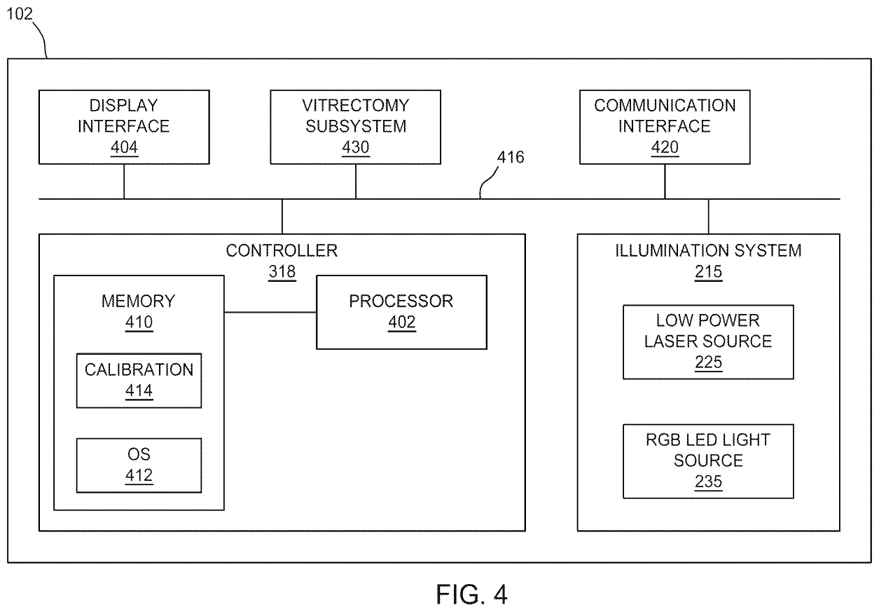

FIG. 4 illustrates a block diagram of selected components of the illumination system 215 (which represents illumination systems 215a-b) shown in FIGS. 2A-2B, in accordance with certain embodiments of the present disclosure. In FIG. 4, the controller 318 is in communicatively coupled to the illumination system 215, which includes the low power laser source 225 and the RGB LED light source 235, a display interface 404, a communication interface 420, and a vitrectomy subsystem 430 (or vitrectomy system) via a bus 416 within the console 102.

The controller 318 includes a processor 402 in communication with a memory 410. In certain embodiments, the controller 318 is configured to interface with the illumination system 215, the vitrectomy subsystem 430, and other components of the console 102 through the bus 416. The controller 318 may also be connected to one or more displays, such as the display 106, via the display interface 404.

The memory 410 may include persistent, volatile, fixed, removable, magnetic, and/or semiconductor media. The memory 410 may be configured to store one or more machine-readable commands, instructions, data, and/or the like. In some implementations, the memory 410 may include one or more sets and/or sequences of instructions, such as an operating system (OS) 412, a calibration application 414, and the like. Examples of the operating system 412 may include, but are not limited to, UNIX (Uniplexed Information Computing System) or UNIX-like operating system, a Windows® family operating system, or another suitable operating system.

The calibration application 414 may be executed by the processor 402 to perform laser or light calibration operations as described herein including, but not limited to, operations related to calibrating an observed output flux or brightness relative to a received setting value, and the like. For example, the calibration application 414 may comprise one or more functions for converting a desired illumination setting (e.g., desired brightness, color, intensity, laser power, etc.), provided by a user, into a calibrated control signal for the low power laser source 225 and/or the RGB LED light source 235. For example, during operation of the illumination system 215, a user may input a desired illumination setting through a user interface of the display 106. The processor 402 then uses the desired illumination setting as input into the one or more functions and outputs a calibrated control signal that is then transmitted to illumination system 215. A drive circuit of the low power laser source 225 and/or the RGB LED light source 235 then converts the calibrated control signal into a corresponding electrical input that is provided to the low power laser source 225 and/or the RGB LED light source 235 to produce the low power laser beam 230 and/or the RGB light 245 corresponding to the desired illumination setting.

Note that although the processor 402 and the memory 410 are shown as components of the controller 318, the illumination system 215 may comprise its own dedicated processor and memory, which may perform the same or similar functions as the processor 402 and the memory 410 described above. That is, the illumination system 215 may include a dedicated processor and memory, and the dedicated memory may include a calibration application configured to perform laser/light calibration operations as described herein. In such embodiments, upon determining one or more calibration functions, the functions may then be stored in memory 410 of controller 318 for use during operation of the illumination system 215.

The vitrectomy subsystem 430 can be a pneumatic system or an electric system configured for driving a vitrectomy probe (e.g., the vitrectomy probe 120). When the vitrectomy subsystem 430 is a pneumatic system, the pneumatic system includes an air compressor, control valves, and a pneumatic drive. The air compressor is disposed in the console 102 and generates compressed air. The compressed air is regulated by the control valves, which adjust a pressure and a flow rate of the compressed air. The air regulated by the control valves is delivered to the vitrectomy probe 120, which contains a pneumatic system. Thus, this mechanism translates the air pressure into mechanical motion, driving cutting action of the vitrectomy probe 120.

When the vitrectomy subsystem 430 is an electric system, the electric system includes an electric motor, control electronics, and a transmission mechanism. The electric motor drives the vitrectomy probe 120. The motor is controlled by electronics within the console 102 that precisely adjust the speed and movement of the motor. The electric motor's movement is transmitted to the cutting mechanism of the vitrectomy probe 120 through a series of gears or direct drive systems.

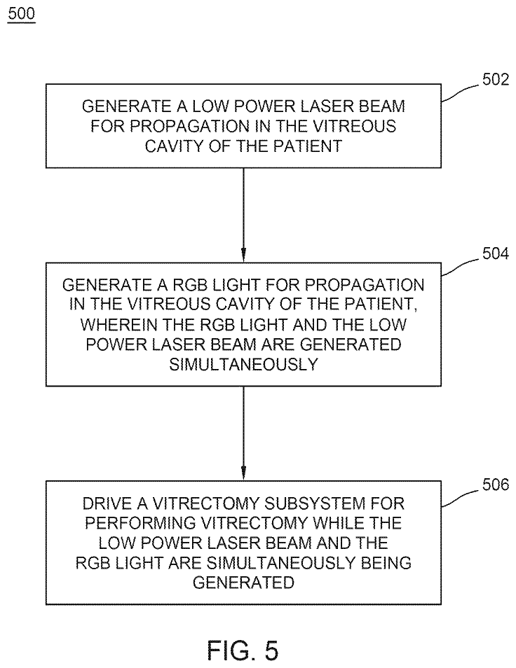

FIG. 5 illustrates a flowchart of a method 500 for illuminating a vitreous cavity of a patient, according to an embodiment. In some embodiments, the method 500 may be performed by the surgical console 102. Note that blocks 502, 504, and 506, may be performed in any order and/or simultaneously and not necessarily with the order presented herein.

At block 502, the method 500 includes generating a low power laser beam (e.g., low power laser beam 230) for propagation in the vitreous cavity of the patient. In some embodiments, the low power laser beam is generated by the low power laser source 225 of the surgical console 102 and is transmitted by one of the first illumination device 130 or the second illumination device 140 into the vitreous cavity.

At block 504, the method 500 includes generating a RGB light (e.g., RGB light 245) for propagation in a vitreous cavity of a patient, wherein the RGB light and the low power laser beam are simultaneously generated. In some embodiments, the RGB light is generated by the RGB LED light source 235 of the surgical console 102 and is transmitted by the other one of the first illumination device 130 or the second illumination device 140 into the vitreous cavity 200.

Note that the RGB light and the low power laser beam being simultaneously generated refers to the RGB light and the low power laser beam being generated or emitted both at the same time for a certain duration of time. In other words, the RGB light and the low power laser beam being simultaneously generated may include (1) initiating generation of the low power laser beam first and then initiating generation of the RGB light while still generating the low power laser beam, such that the low power laser beam and the RGB light are both generated at least for a certain duration of time (e.g., during at least part of the vitrectomy procedure), (2) initiating generation of the RGB light first and then initiating generation of the low power laser beam while still generating the RGB light, such that the low power laser beam and the RGB light are both generated at least for a certain duration of time (e.g., during at least part of the vitrectomy procedure), or (3) initiating generation of the RGB light and the low power laser beam at the same time and continuing to generate both the RGB light and the low power laser beam at least for a certain duration of time (e.g., during at least part of the vitrectomy procedure).

At block 506, driving (i.e., operating) a vitrectomy subsystem (e.g., vitrectomy subsystem 430) for performing vitrectomy while the low power laser beam and the RGB light are simultaneously being generated. The vitrectomy subsystem 430 can be used with the vitrectomy probe 120 to remove the vitreous 202 from the vitreous cavity 200. When the low power laser beam and the RGB light are simultaneously generated and transmitted into the vitreous cavity 200, for example, the combined colored low power laser beam is absorbed (or picked up) by collagen fibers in the vitreous 202, thereby further illuminating the vitreous 202 and making it more visible to the surgeon 105. Thus, the surgeon 105 is able to more efficiently remove the vitreous 202 from the vitreous cavity 200 due to the improved visualization of the vitreous 202.

The present disclosure may be embodied in other specific forms without departing from its spirit or essential characteristics. The described embodiments are to be considered in all respects only as illustrative and not restrictive. The scope of the present disclosure is, therefore, indicated by the appended Claims rather than by this Detailed Description. All changes which come within the meaning and range of equivalency of the Claims are to be embraced within their scope.

Reference throughout this specification to features, advantages, or similar language does not imply that all the features and advantages that may be realized with the present disclosure should be or are in any single embodiment of the disclosure. Rather, language referring to the features and advantages is understood to mean that a specific feature, advantage, or characteristic described in connection with an embodiment is included in at least one embodiment of the present disclosure. Thus, discussions of the features and advantages, and similar language, throughout this specification may, but do not necessarily, refer to the same embodiment.

Furthermore, the described features, advantages, and characteristics of the disclosure may be combined in any suitable manner in one or more embodiments. One skilled in the relevant art will recognize, in light of the description herein, that the disclosure can be practiced without one or more of the specific features or advantages of a particular embodiment. In other instances, additional features and advantages may be recognized in certain embodiments that may not be present in all embodiments of the present disclosure.

Reference throughout this specification to “one embodiment”, “an embodiment”, or similar language means that a particular feature, structure, or characteristic described in connection with the indicated embodiment is included in at least one embodiment of the present disclosure. Thus, the phrases “in one embodiment”, “in an embodiment”, and similar language throughout this specification may, but do not necessarily, all refer to the same embodiment.

The foregoing description is provided to enable any person skilled in the art to practice the various embodiments described herein. Various modifications to these embodiments will be readily apparent to those skilled in the art, and the generic principles defined herein may be applied to other embodiments. Thus, the claims are not intended to be limited to the embodiments shown herein but are to be accorded the full scope consistent with the language of the claims.

What is claimed is:

1. An ophthalmic system comprising:
a low power laser source configured to generate a low power laser beam, wherein the low power laser source has a power of between 5 milliwatts (mW) and 20 mW; and
a first illumination device configured to transmit the low power laser beam into a vitreous cavity of an eye.

2. The ophthalmic system of claim 1 further comprising:
a red-green-blue (RGB) light-emitting diode (LED) light source configured to generate a RGB light; and
a second illumination device configured to transmit the RGB light into the vitreous cavity of the eye.

3. The ophthalmic system of claim 2, wherein the first illumination device and the second illumination device are the same device.

4. The ophthalmic system of claim 2, wherein:
the first illumination device is a chandelier; and
the second illumination device is an endoilluminator.

5. The ophthalmic system of claim 2, wherein:
the first illumination device is an endoilluminator; and
the second illumination device is a chandelier.

6. The ophthalmic system of claim 1, wherein the first illumination device comprises a diffusing surface at a distal end, the diffusing surface configured to diffuse the low power laser beam.

7. The ophthalmic system of claim 1, wherein the first illumination device comprises a diffusing lens at a distal end, the diffusing lens configured to diffuse the low power laser beam.

8. The ophthalmic system of claim 1, wherein:
the ophthalmic system is a surgical console, and
the ophthalmic system further comprises a vitrectomy system configured to drive a vitrectomy probe while an RGB light and the low power laser beam are being simultaneously generated.

9. The ophthalmic system of claim 1, wherein the ophthalmic system is an illumination system.

10. A method for illuminating a vitreous cavity of a patient, comprising:
generating a low power laser beam for propagation in the vitreous cavity of the patient;
generating a red-green-blue (RGB) light for propagation in the vitreous cavity of the patient, wherein the RGB light and the low power laser beam are generated simultaneously; and
driving a vitrectomy subsystem for performing vitrectomy while the low power laser beam and the RGB light are simultaneously being generated.

11. The method of claim 10, wherein the low power laser beam is generated by a low power laser source that has a power of between 5 milliwatts (mW) and 20 mW.

12. The method of claim 10, wherein the low power laser beam and the RGB light are transmitted by a single illumination device.

13. The method of claim 10, wherein:
the low power laser beam is transmitted by a first illumination device; and
the RGB light is transmitted by a second illumination device.

14. The method of claim 10, wherein:
the low power laser beam is transmitted by a chandelier; and
the RGB light is transmitted by an endoilluminator.

15. The method of claim 10, wherein:
the low power laser beam is transmitted by an endoilluminator; and
the RGB light is transmitted by a chandelier.
